(19) United States
(12) Patent Application Publication
(10) Pub. No.: US 2026/0273166 A1
(43) Pub. Date: Sep. 17, 2026

(54) Dual Lumen Negative Pressure Wound Therapy System With Continuous Leak at the Wound Site

(71) Applicant: DeRoyal Industries, Inc., Powell, TN (US)

(72) Inventors: Dhanvin S. Desai, Knoxville, TN (US); Thomas Paul Lawhorn, Sevierville, TN (US); Jonathan M. Cayce, Knoxville, TN (US); Ethan Valentine, Knoxville, TN (US)

(73) Assignee: DeRoyal Industries, Inc., Powell, TN (US)

(21) Appl. No.: 19/564,446

(22) Filed: Mar. 12, 2026

Related U.S. Application Data
(60) Provisional application No. 63/770,606, filed on Mar. 12, 2025.

(51) Int. Cl. A61M 1/00

(52) U.S. Cl. CPC A61M 1/962; A61M 1/964; A61M 2202/0014; A61M 2202/04; A61M 2205/3327; A61M 2205/3344; A61M 2205/3382; A61M 2205/50

(57) ABSTRACT

A system for managing reduced pressure to a wound site including a wound enclosure configured to form a substantially sealed volume around the wound site. A reduced pressure source is in fluid communication with the wound enclosure and is operable to apply reduced pressure to the wound enclosure for delivering exudate collected from the wound site to a canister through a first lumen. A secondary pressure source is operable to apply a secondary pressure to the wound enclosure through a second lumen to facilitate the flow of the exudate from the wound site to the canister through the first lumen. A vent is disposed adjacent the wound site that is open to the atmosphere for introducing ambient air into the wound enclosure through the vent.

### CROSS REFERENCE TO RELATED APPLICATION

This application claims priority to co-pending U.S. Provisional Application Ser. No. 63/770,606 filed Mar. 12, 2025, entitled “Dual Lumen Negative Pressure Wound Therapy System with Controlled Leak at the Wound Dressing,” the entire contents of which is incorporated herein by reference.

### FIELD

This disclosure relates to the field of negative pressure wound therapy. More particularly, this disclosure relates to a system for managing the reduced pressure applied to a sealed wound enclosure at the wound site for delivering exudate from the wound site to a canister.

### BACKGROUND

The primary purpose of negative pressure wound therapy (also referred to as “reduced pressure therapy” or “vacuum therapy” in the medical community) is to apply a reduced pressure (also referred to as “negative pressure” or “vacuum pressure” but will be referred to herein as “reduced pressure”) to a wound site to stimulate healing and remove excess exudate from the wound site. The major components of a negative pressure wound therapy system (hereinafter “NPWT system”) include a reduced pressure source (e.g., vacuum pump), a waste canister for collecting extracted exudate from the wound site, a wound enclosure for covering the wound site (e.g., an assembly typically having various layers such as a manifold, drape, dressing, and dome for connecting the enclosure to the reduced pressure tubing, etc.), and one or more lumens (e.g., tubing) fluidly connecting the wound enclosure to the reduced pressure source and the canister.

One issue encountered by NPWT systems is maintaining a consistent and precise prescribed pressure at the wound site over the course of a treatment. Various situations can cause this inconsistency and impreciseness. Common examples include the viscosity of the exudate being removed (i.e., changes in viscosity during treatment and/or exudate having greater viscosities), different desired flow rates of the exudate being removed, blockage within the tubing, bolus conditions at the wound, etc.

What is needed, therefore, is an improved NPWT system for managing reduced pressure being delivered to a wound site including enhancing consistency and precision of the prescribed pressure.

### SUMMARY

The above and other needs are met by a system for managing reduced pressure to a wound site having a wound enclosure configured to form a substantially sealed volume around the wound site. A reduced pressure source is in fluid communication with the wound enclosure and is operable to apply reduced pressure to the wound enclosure for delivering exudate collected from the wound site to a canister through a first lumen. A secondary pressure source is in fluid communication with the wound enclosure and is operable to apply a secondary pressure to the wound enclosure through a second lumen to facilitate the flow of the exudate from the wound site to the canister through the first lumen. A vent is disposed adjacent the wound site that is open to the atmosphere for introducing ambient air into the wound enclosure through the vent.

According to certain embodiments, the wound enclosure includes an enclosure interface having a dome and a flange disposed around the dome. The first lumen and the second lumen are fluidly connected to the dome and the vent is disposed in the flange.

According to certain embodiments, the vent is operable to introduce a continuous flow of ambient air into the would enclosure. In some embodiments, the system further includes a controller configured to communicate with the secondary pressure source for selectively activating the secondary pressure source to apply the secondary pressure to the wound enclosure while the vent introduces the continuous flow of ambient air into the wound enclosure.

According to certain embodiments, the system further includes a pump unit that includes the reduced pressure source and the secondary pressure source. In some embodiments, the secondary pressure source includes a valve that is configured to be selectively opened to the atmosphere for applying the secondary pressure to the wound enclosure.

According to some embodiments, the pump unit further includes a first pressure sensor for measuring a pressure of a fluid flow along a first fluid flow path that includes the first lumen and a second pressure sensor for measuring a pressure of a fluid flow along a second fluid flow path that includes the second lumen. In some embodiments, a controller is operable to receive and compare pressure readings from the first pressure sensor and the second pressure sensor and selectively activate the secondary pressure source based on the comparison of the pressure readings. In some embodiments, the controller is operable to receive and compare pressure readings from the first pressure sensor and the second pressure sensor and determine an operating condition of the system selected from a group consisting of a blockage condition, a canister fill condition, and a bolus condition.

According to another embodiment of the disclosure, a system for managing reduced pressure to a wound site includes a wound enclosure configured to form a substantially sealed volume around the wound site, a pump unit, and a vent. The pump unit includes a reduced pressure source, a secondary pressure source, a first pressure sensor, a second pressure sensor, and a controller. The reduced pressure source is in fluid communication with the wound enclosure along a first fluid flow path and is operable to apply reduced pressure to the wound enclosure for delivering exudate collected from the wound site to a canister through a first lumen. The secondary pressure source is in fluid communication with the wound enclosure along a second fluid flow path and is operable to apply a secondary pressure to the wound enclosure through a second lumen to facilitate the flow of the exudate from the wound site to the canister through the first lumen. The first pressure sensor is in fluid communication with the first fluid flow path for measuring a pressure along the first fluid flow path. The second pressure sensor is in fluid communication with the second fluid flow path for measuring a pressure along the second fluid flow path. The controller is operable to receive and compare pressure readings from the first pressure sensor and the second pressure sensor and selectively activate the secondary pressure source based at least in part on the comparison of the pressure readings. The vent is disposed adjacent the wound site that is open to the atmosphere for introducing a continuous flow of ambient air into the would enclosure.

According to certain embodiments, the wound enclosure includes an enclosure interface having a dome and a flange disposed around the dome with the first lumen and the second lumen fluidly connected to the dome and the vent being disposed in the flange.

According to certain embodiments, the controller is configured to communicate with the secondary pressure source for selectively activating the secondary pressure source to apply the secondary pressure to the wound enclosure while the vent introduces the continuous flow of ambient air into the wound enclosure.

According to certain embodiments, the secondary pressure source includes a valve that is configured to be selectively opened to the atmosphere by the controller for applying the secondary pressure to the wound enclosure.

According to certain embodiments, the controller is operable to determine an operating condition of the system based at least in part on the comparison of the pressure readings from the fist pressure sensor and the second pressure sensor, the operating condition selected from a group consisting of a blockage condition, a canister fill condition, and a bolus condition.

According to another embodiment of the disclosure, a method of delivering reduced pressure to a wound site includes: applying reduced pressure to a wound enclosure for delivering exudate collected from the wound site to a canister through a first lumen; providing a secondary pressure source in fluid communication with the wound enclosure that is operable to apply a secondary pressure to the wound enclosure through a second lumen to facilitate the flow of the exudate from the wound site to the canister through the first lumen when the secondary pressure source is activated; and continuously introducing ambient air into the wound enclosure through a vent disposed adjacent the wound site.

According to certain embodiments, the secondary pressure source is operable to be selectively activated to apply the secondary pressure to the wound enclosure while the vent is continuously introducing ambient air into the wound enclosure.

According to certain embodiments, the method further includes providing a pump unit wherein the pump unit includes the reduced pressure source and the secondary pressure source. According to some embodiments, the secondary pressure source includes a valve that is configured to be selectively opened to the atmosphere for applying the secondary pressure to the wound enclosure.

According to certain embodiments, the method further includes determining a pressure of a first fluid flow path that includes the first lumen; determining a pressure of a second fluid flow path that includes the second lumen; determining a pressure differential between the pressure of the first fluid flow path and the pressure of the second fluid flow path; and selectively activating the secondary pressure source based on the determination of the pressure differential between the first fluid flow path and the second fluid flow path. In some embodiments, the method further includes determining an operating condition of the system selected from a group consisting of a blockage condition, a canister fill condition, and a bolus condition based at least in part on the pressure differential between the pressure of the first fluid flow path and the pressure of the second fluid flow path.

### BRIEF DESCRIPTION OF THE DRAWINGS

Other embodiments of the disclosure will become apparent by reference to the detailed description in conjunction with the figures, wherein elements may not be to scale so as to more clearly show the details, wherein like reference numbers indicate like elements throughout the several views, and wherein:

FIG. 1 depicts a simplified schematic of a dual lumen NPWT system according to one embodiment of the prior art.

FIG. 2 depicts a simplified schematic of a dual lumen NPWT system having a vent disposed adjacent the wound site for providing a continuous leak of atmospheric air to the wound enclosure according to one embodiment of the present disclosure.

FIGS. 3-5 depicts research results evidencing the benefits of a dual lumen NPWT system that utilizes a continuous leak disposed adjacent the wound site.

### DETAILED DESCRIPTION

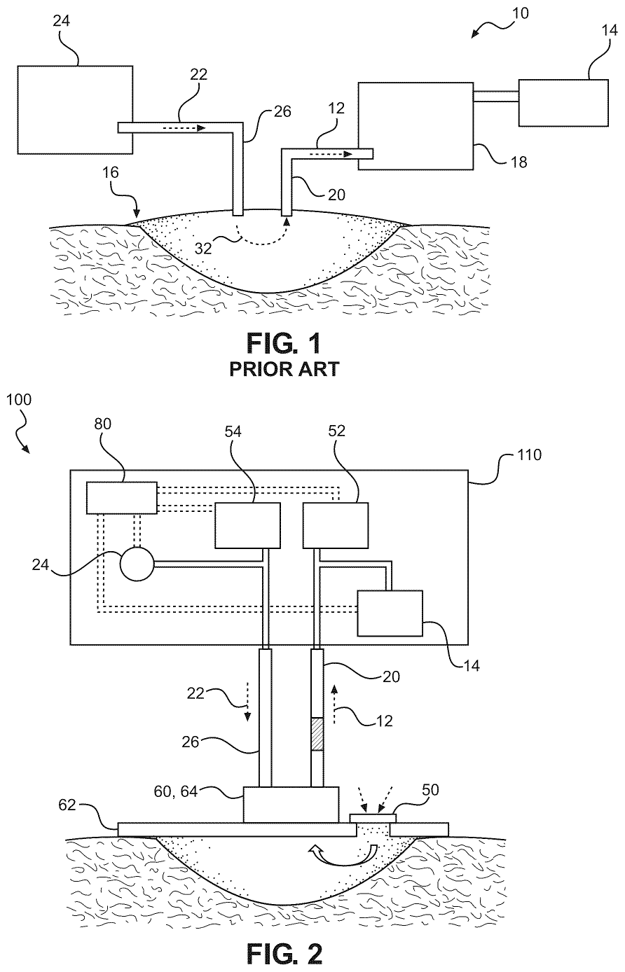

Referring initially to FIG. 1, an exemplary NPWT system 10 as known in the art is depicted having two fluidly connected flow paths. According to a first fluid flow path (indicated by arrow 12), a reduced pressure source 14 is operable to provide a reduced pressure to a wound enclosure 16 such that fluid flows from the wound enclosure 16 to canister 18 through first lumen 20 (also referred to herein as a reduced pressure lumen 20). The reduced pressure generated by the reduced pressure source 14 (i.e., vacuum pump) serves both to stimulate healing by applying the reduced pressure to the wound site and removing exudate from the wound enclosure 16 to the canister 18. According to a second fluid flow path (indicated by arrow 22), a secondary pressure source 24 is operable to provide a secondary pressure to the wound enclosure 16 through second lumen 26 (also referred to herein as a secondary pressure lumen). Using the second fluid flow path 22, the reduced pressure being applied to the wound enclosure 16 by the reduced pressure source 14 may be measured/monitored as further described below. Further, the second fluid flow path 22 may also be connected to the secondary pressure source 24 to provide a secondary pressure to the wound enclosure 16 through the second lumen 26 to assist the reduced pressure source as needed/desired.

The secondary pressure provided by the secondary pressure source 24 will be greater than the pressure at the wound enclosure 16 to prevent exudate from the wound site entering the second lumen 26. Similarly, the secondary pressure will also be greater than the reduced pressure generated by the reduced pressure source 14 within the first lumen 20. Accordingly, as the second fluid flow path 22 is fluidly connected to the first fluid flow path 12 via the wound enclosure 16 (including a dome/tubing connector disposed at the entrance to the wound enclosure 16) and the pressure of the second fluid flow path 22 is greater than the pressure of the first fluid flow path 12, air from the second fluid flow path 22 is pushed into the first lumen 20 (as indicated by arrow 32 and referred to herein as a “converged fluid flow path”) when both the reduced pressure source 14 and secondary pressure source 24 are activated. The converged fluid flow path 32 may be used to facilitate flow of the exudate from the wound site to the canister 18 through the first lumen 20. In typical embodiments, the reduced pressure source 14 settings are configured to deliver a reduced pressure treatment level of about −125 mmHg to the wound enclosure 16 via first lumen 20 while the secondary pressure source 24 is activated to help maintain the same treatment level by facilitating flow of exudate from the wound site to the canister through the first lumen 20.

The secondary pressure provided by the secondary pressure source 24 to the second lumen 26 may come from various sources. For example, the secondary pressure may be delivered from the outlet of the reduced pressure source 14, a secondary pump within the pump unit containing the reduced pressure source 14, or a filtered valve open to the atmosphere. In preferred embodiments, the secondary pressure source 24 is a filtered valve included in the pump unit containing the reduced pressure source 14 that is in fluid communication with the second lumen 26 and is able to be selectively opened to the atmosphere for providing the secondary pressure to the second lumen 26.

In certain embodiments, the first lumen 20 and second lumen 26 are combined in a more traditional dual lumen system as known in the art and described in, for example U.S. Patent Publication No. 2020/0086017, the entire contents of which being incorporated herein in its entirety. In certain embodiments, and for purposes of the present disclosure, the dual lumen system may also be incorporated into a multi-lumen system in which the first fluid flow path 12 and/or second fluid flow path 22 are formed from multiple lumens that converge or otherwise fluidly connect at a connector within the dual lumen system and/or at the connection to the wound enclosure. For example, a multi-lumen system having tubing containing a central first lumen 20 fluidly connected to the reduced pressure source 14 with multiple second lumens 26 disposed around the first lumen 20 and each fluidly connected to the secondary pressure source 24 would, for purposes of the present disclosure, be considered a dual lumen system as it includes at least a first lumen 20 for delivering reduced pressure through the first fluid flow path 12 and at least a second lumen 26 for delivering secondary pressure through the second fluid flow path 22.

With reference to FIG. 2, an exemplary NPWT system 100 of the present disclosure is depicted having pump unit 110 (note the canister 18 is omitted from FIG. 2 for simplicity but would be similarly positioned between the reduced pressure source 14 and first lumen 20 along the first fluid flow path 12 as depicted in FIG. 1). Pump unit 110 includes the reduced pressure source 14 and secondary pressure source 24. According to this embodiment, the secondary pressure source 24 is preferably a valve included in the NPWT pump unit 110 that is fluidly connected to the second lumen 26 and able to be selectively opened to the atmosphere for introducing ambient air to the second fluid flow path 22. As also depicted in FIG. 2, system 100 includes a dome interface 60 for fluidly connecting the first lumen 20 and second lumen 26 to the wound enclosure 16.

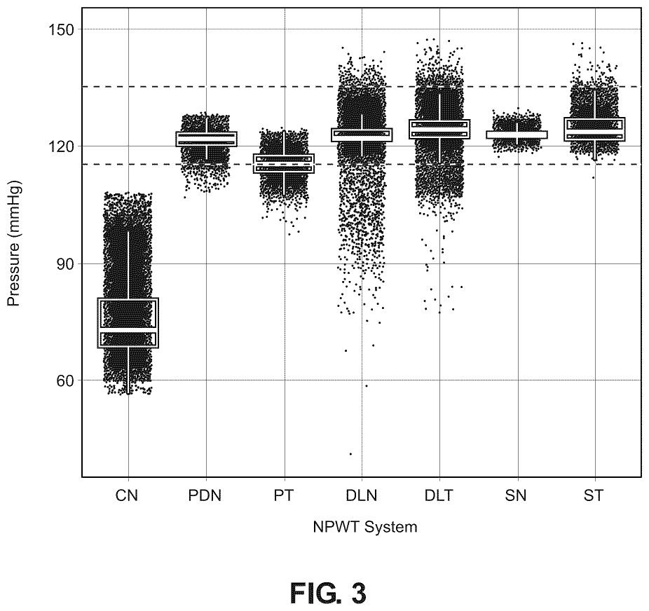

With continued reference to FIG. 2, system 100 includes a vent 50 disposed adjacent the wound site for introducing a continuous leak of atmospheric air to the wound enclosure 16. For purposes of the present disclosure, the vent 50 being “disposed adjacent the wound site” refers to the vent 50 being positioned distally from the pump unit 110 and in close proximity to the wound enclosure 16. In preferred embodiments, air introduced by vent 50 would enter the system as part of the converged fluid flow path 32 without ever being included in the first fluid flow path 12. For example, the dome interface 60 for connecting the first lumen 20 and second lumen 26 to the wound enclosure 16 may include the vent 50 in the flange 62 of the dome interface 60 as depicted schematically in FIG. 2. Alternatively, the vent 50 may be positioned in the dome 64 of the dome interface 60 or otherwise fluidly connected to the dome 64 instead of the flange 62 surrounding the dome 64. In yet another alternative embodiment, the vent 50 is fluidly connected to the first lumen 12 adjacent the dome interface 60. As evidenced in the below described test results of FIGS. 3-5, a continuous flow of filtered air from the vent 50 disposed adjacent the wound site allows the dual lumen system described above with respect to FIG. 1 to work more efficiently and provides improved consistency/precision in delivering prescribed NPWT treatment pressures to a wound enclosure.

According to certain embodiments, the vent 50 disposed adjacent the wound site is operable to provide a continuous leak of atmospheric air at a flow rate of about 10-70 mL/min depending on the pressure in the system. However, it is believed flow rates outside this range could also be used and still be effective.

With continued reference to FIG. 2, the continuous leak adjacent the wound site is intended as a continuous flow of filtered air to facilitate fluid flow and help manage pressure in the first lumen 20. On the other hand, as noted above, the secondary pressure source 24 fluidly connected to the second lumen 26 (e.g., pump unit 110 having a valve to the atmosphere that is fluidly connected to second lumen 26) is preferably able to be selectively activated to deliver the secondary pressure to the second lumen 26 as needed or desired. For example, as further described below, and according to certain embodiments, the system may selectively activate the secondary pressure 24 (e.g., open and closing the valve to the atmosphere) according to predetermined and periodic purging cycles, alarm checks, when blockage is detected, etc. In other embodiments, the secondary pressure source 24 may remain activated during certain prescribed treatment sessions to provide improved consistency precision in delivering the reduced pressure as evidenced by the test results described herein.

According to another aspect of the embodiment as depicted in FIG. 2, the system 100 preferably includes a first pressure sensor 52 disposed along the first fluid flow path 12 between the wound enclosure 16 and the reduced pressure source 14. In preferred embodiments, the first pressure sensor 52 is preferably included as part of the pump unit 110 such that it is disposed between the canister 18 and the reduced pressure source 14 for measuring the pressure of the fluid flow from the enclosure 16 through the canister 18. A second pressure sensor 54 is preferably also included as part of the pump unit 110 such that it is disposed along the second fluid flow path 22 between the secondary pressure source 24 and the wound enclosure 16 for measuring the pressure in the second lumen 22 and/or at the wound enclosure 16. In this regard, a pressure measured at one end of a lumen is substantially equal to the pressure at the other end of the lumen so long as very little to no liquid/exudate is present in the lumen and the lumen inside diameter to length ratio is not too small. As result, when the secondary pressure source 24 is inactive, or the second lumen 26 is otherwise fluidly isolated from any secondary pressure generated from the secondary pressure source 24 (e.g., the valve to the atmosphere at the pump unit 110 is in a closed position), the second pressure sensor 76 may be used to measure the reduced pressure being applied to the wound enclosure 16 by the reduced pressure source 14.

On the other hand, when the reduced pressure source 14 and secondary pressure source 24 are both activated, various operating conditions may be determined by comparing the pressure readings from the first pressure sensor 52 with the pressure readings from the second pressure sensor 54. In particular, the monitoring of the pressure readings from the two pressure sensors 52, 54 may provide information regarding the amount of exudate being delivered from the wound enclosure 16 to the canister 18 via first lumen 20. For example, a small difference between the readings of the pressure sensors 52, 54 indicates a small amount of exudate is being delivered to the canister via the first lumen 20 while a greater difference between the pressure readings indicates a larger amount of exudate in the first lumen 20. In this regard, when the pressures of the first fluid flow path 12 and second fluid flow path 22 are the same or substantially similar during activation of both the reduced pressure source 14 and the secondary pressure source 24, this indicates that there should be a relatively small amount of exudate (if any) flowing from the wound enclosure 16 to the canister because the secondary pressure from the second fluid flow path 22 has been able to converge with the reduced pressure from the first fluid flow path 12 and the air from the vent 50 to form a converged fluid flow path having a similar pressure along the entirety of the converged path. However, when there is a larger amount of exudate in the first lumen 20, the pressure of the first fluid flow path 12 should be substantially different than the pressure of the second fluid flow path 22 because the second fluid flow path 22 was not able to easily converge with the first fluid flow path 12 due to the existence of significant exudate in the first lumen 20. Depending on how great the difference between the pressure readings, it might also be determined that (1) a blockage condition exists in the first lumen 20 or second lumen 22 and/or (2) a fill condition exists in the canister 18 indicating that the canister 18 is close to being filled with exudate.

In preferred embodiments, and with continued reference to FIG. 2, NPWT system 100 further includes a controller 80 configured to communicate with at least the reduced pressure source 14, secondary pressure source 24, the first pressure sensor 52, and the second pressure sensor 54. According to this embodiment, controller 80 is configured to compare the pressure readings from the first and second pressure sensors 52, 54 to determine when to activate the secondary pressure source 24. For example, secondary pressure source 24 may initially be set to activate according to predetermined time intervals and/or for a predetermined amount of time during a purge cycle. The secondary pressure source 24 continues to activate according to the predetermined time intervals and/or predetermined amount of time so long as the pressure differential between the first fluid flow path 12 as determined by first pressure sensor 52 and the second fluid flow path 22 as determined by second pressure sensor 54 stays below a minimum threshold (with assistance from the vent 50). However, when the minimum threshold for the pressure differential is exceeded, the controller 80 may be configured to activate the secondary pressure source 24 more frequently and/or for a greater amount of time until the pressure differential returns to below the minimum threshold.

Intermittent and/or controlled activation of the secondary pressure source 24 while maintaining the continuous leak provided by vent 50 disposed adjacent the wound site would provide further benefits and potential applications in addition to assisting in quick removal of the exudate from the wound enclosure 16 through first lumen 20 as described above. For example, it has been observed that varying the reduced pressure at the wound enclosure 16 enables the extraction of more exudate than when the reduced pressure is kept constant. Providing a controllable secondary pressure from secondary pressure source 24 permits variance of the reduced pressure delivered to the wound enclosure 16 by the reduced pressure source 14 while the constant leak provided by vent 50 helps maintain the flow of exudate through the first lumen 20 during times when the secondary source 24 is not activated.

According to certain embodiments, the system 100 is designed to adjust the flow “setting” of the secondary pressure source 24 by varying the time the secondary pressure source 24 is activated to allow for different volumes of secondary pressure into the system. For example, in the case of a situation in which it is determined that the secondary pressure source 24 is needed to be activated, the activation duration may be proportional to the needed change in pressure as determined by the controller 80. Similarly, the duration in which the secondary pressure source is activated may be an adjustable setting for different detection algorithms. For example, when a blockage is detected, the secondary pressure source 24 may be activated for a preset blockage time-period while detection of a canister full condition may activate the secondary pressure source 24 for a different canister full time-period. In most embodiments, the time in which the secondary pressure source is activated typically ranges from 100 ms to 2000 ms per opening.

According to certain embodiments, when a bolus condition is detected, the secondary pressure source 24 may remain activated while the reduced pressure source 14 runs continuously to allow for rapid exudate extraction from the wound enclosure 16 that is further facilitated by the continuous leak provided by the vent 50 disposed adjacent the wound site. Similarly, in systems in which instillation fluid is delivered from an instillation fluid source, the dual lumen system in combination with the continuous leak may assist in movement of the installation fluid during the known instillation therapy where there are regular and known boluses of fluid.

With reference to FIGS. 3-5, research results shown evidencing the benefits of a dual lumen NPWT system 100 that utilizes a continuous leak disposed adjacent the wound site. The benefits include consistency and preciseness of the reduced pressure being delivered during a treatment session in which a constant pressure is intended to be maintained. The dual lumen system 100 allows for control of the reduced pressure delivered to the wound enclosure 16 by sensing the pressure delivered to the wound enclosure 16 through the second lumen 26 as described above. The research shows how a dual lumen NPWT system with a continuous leak provided by vent 50 disposed adjacent the wound site improves the accuracy and precision of NPWT treatment delivered to the wound enclosure 16 under different operating variables/exudating conditions. The variables tested included (1) viscosity of the exudate with normal exudate viscosity conditions of about 1.158 cP and thick viscosity conditions of about 35.5 cP; (2) exudate removal rate under a normal exudating rate of about 4 cc per hour and high exudating rate of about 50 cc per hour; and (3) starting with a bolus condition to monitor how exudate in a simulated wound enclosure impacted pressure management. Data was analyzed by assuming a normal distribution and reporting the 99.7% confidence interval that contains all data.

FIG. 3 shows results of a first test of different NPWT systems at an exudating rate of 4 cc per hour using normal and thick viscosity with a negative pressure setting of 125 mmHg. A 4 cc condition represents the maximum exudating rate a patient can go home and receive therapy. Data in the box plot represents three trials of each of the following NPWT systems for at least 8 hours: “CN” refers to a closed single-lumen system (having only a reduced pressure source) with a normal exudate viscosity; “PDN” refers to a single-lumen system with a continuous leak adjacent the wound site and a normal exudate viscosity; “PT” refers to a single-lumen system with a continuous leak and a thick exudate viscosity; “DLN” refers to a dual lumen system (the pump unit having both a reduced pressure source and a secondary pressure source) with no leak at the wound site and a normal exudate viscosity; “DLT” refers to a dual lumen system with no leak at the wound site and a thick exudate viscosity; “SN” refers to a dual lumen system with a continuous leak and a normal exudate viscosity; and “ST” refers to a dual lumen system with a continuous leak and a thick exudate viscosity.

The results shown in FIG. 3 are summarized as follows: (1) The closed single-lumen system (CN) demonstrates that it cannot manage pressure appropriately as a leak in the dressing is needed to help move fluid even with a normal exudate viscosity (75.63 mmHg±30.59 mmHg; 0% within ±10 of desired 125 mmHg setting); (2) the single-lumen system with a continuous leak shows similar pressure management with normal exudate viscosity (PDN: 121.65 mmHg±6.94 mmHg; 99% within±10 of 125 mmHg setting) as the dual lumen system without an additional leak at the wound site (DLN: 122.78 mmHg±14.69 mmHg, 97% within±10 of 125 mmHg setting); (3) the dual lumen system with a continuous leak with a normal exudate viscosity (SN: 122.89 mmHg±3.63 mmHg; 100% within±10 of 125 mmHg setting) provides improved precision as compared to the other NPWT system conditions with normal exudate viscosity removal; (4) inadequate pressure management occurs with a thick exudate viscosity in the single lumen system with a continuous leak (PT: 115.29 mmHg±8.65 mmHg; only 53% within±10 of 125 mmHg setting); (5) the dual lumen system without an additional leak at the wound site applied pressure with a thick exudate viscosity (DLT: 123.95 mmHg±14.16 mmHg at the 125 mmHg setting) at a more inconsistent range as compared to the dual lumen system with a continuous leak (ST: 124.36 mmHg±10.50 mmHg at the 125 mmHg setting); (6) the dual lumen system with a continuous leak and a thick exudate viscosity (ST) maintained pressure within ±10 of 125 mmHg setting 99.6% of the time compared to 95% for the DLT system test; and (7) in both normal and thick exudate viscosity conditions, the dual lumen system with a continuous leak maintained the pressure with increased precision compared to the dual lumen system without an additional leak at the wound site.

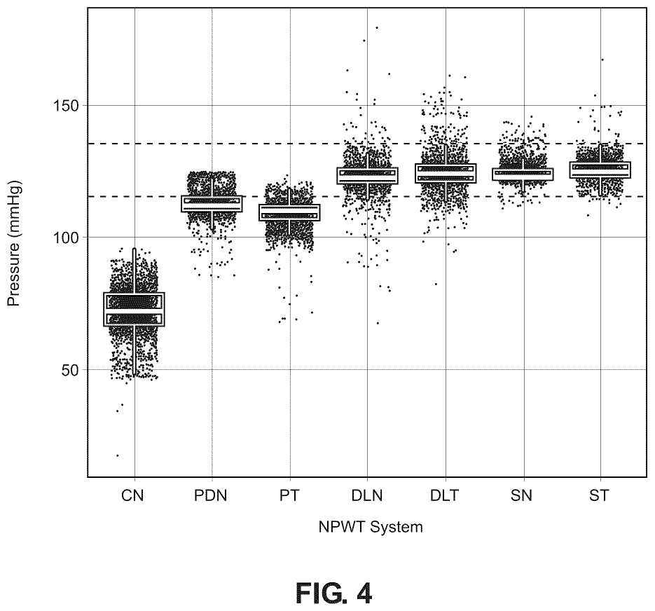

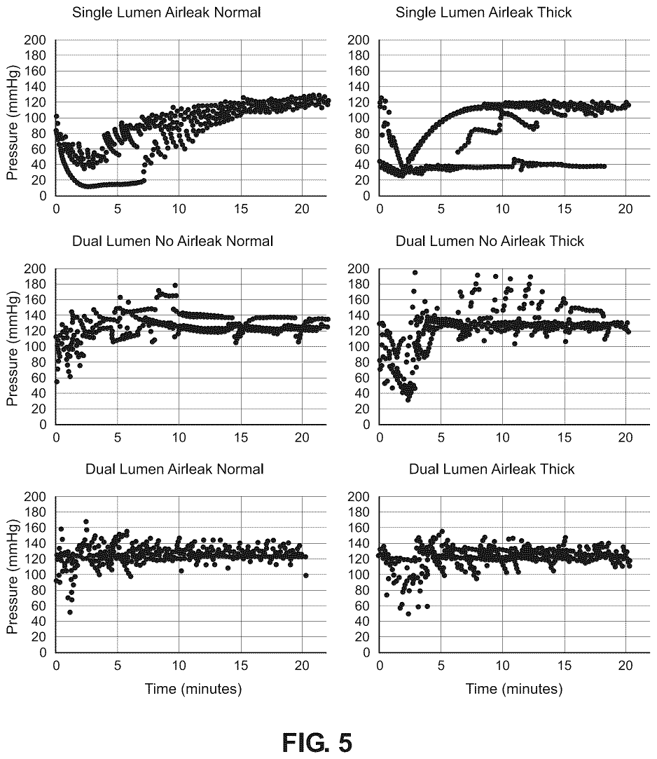

FIG. 4 displays the results from testing the NPWT systems described above at 50 cc per hour with normal and thick exudate. The single lumen systems under testing conditions—(CN), (PDN) and (PDT)—could not maintain an average pressure within 125±10 mmHg. The dual lumen systems without a continuous leak maintained an average pressure within the acceptable range (DLN at 122.69 mmHG±19.66 mmHg and DLT at 124.51 mmHG±21.9 mmHg) but with less precision than the dual lumen systems with the continuous leak (SN at 124.00 mmHg±12.63 mmHg and ST at 125.40 mmHg±13.71 mmHg). The improved precision allowed the dual lumen system with a continuous leak to achieve expected accuracy of at least 95% across the three trials for both normal exudate and thick exudate compared to 92% accuracy for the dual lumen system without an additional leak at the wound site for normal exudate and 86% accuracy for the dual lumen system without an additional leak for thick exudate.

FIG. 5 depicts the results of the time course of three trials involving the systems and testing conditions described above with a dressing being pre-filled with 50 cc of exudate (“Normal”) followed by 100 cc of exudate (“Thick”) injected into the dressing to represent situations in which a bolus of fluid may need to be rapidly removed to allow for the establishment of NPWT (e.g., at initial dressing placement or after the instillation of fluid into a NPWT dressing). The results demonstrate that a single-lumen system, even with an air leak, takes at least 10 minutes to clear and start applying the prescribed pressure. In both dual lumen systems, pressure application at therapy occurs between 2-4 minutes. However, the dual lumen system with a continuous leak reduces the variability in pressure applied for both normal and thick exudating conditions. Note that the thick exudate noticeably increased the magnitude of pressures applied to the wound enclosure for the dual lumen system with no leak.

Overall, the data on pressure management in the exemplary conditions described herein shows how combining a continuous leak from a vent disposed adjacent the wound site with a dual lumen NPWT system improves pressure management at the wound enclosure. Increased accuracy/precision of pressure applied to the wound enclosure ensures the patient receives the prescribed pressure and helps to ensure clinician expectations are met. More specifically, pressure management is improved with the continuous movement of fluid through the reduced pressure lumen to the canister. This is particularly important with thicker exudates as the fluid is heavy enough that a larger pressure differential needs to exist in the lumen for it to continuously move (thicker exudate can need as high as ~100-110 mmHg differential to move up a 3 ft head height). The continuous leak accomplishes this while not requiring the secondary pressure source/valve to be activated as frequently. The continuous leak also prevents backflow of exudate into the wound enclosure by facilitating continuous movement of fluid in the reduced pressure lumen towards the canister.

Additionally, pressure management is improved in that combining the continuous leak adjacent the wound site with a dual lumen system leads to more tightly controlled pressure across the system. In this regard, when designing control algorithms for NPWT systems, the lack of a continuous and constant resistance for the proportional-integral-derivative controller makes the fine tuning of the delivery of reduced pressure difficult. While a dual lumen system helps in this regard by allowing for negative input (i.e., delivery of secondary pressure) in case the system drives pressure too far, the system must trigger this negative input after it has determined that the secondary pressure is needed. This often creates a situation where oscillation in pressure occurs when the system is trying to maintain a tight pressure tolerance. In the system of the present disclosure, the continuous introduction of secondary pressure from the air leak at the wound site creates a smoothing effect on the control system, allowing it to maintain the desired tighter pressure tolerances. Relatedly, activation of the secondary pressure source (e.g., opening the valve to the atmosphere at the pump unit) creates instances of volatility within the system (e.g., ~300-500 ms) in which algorithm decisions cannot be made by the controller. The continuous leak at the wound results in fewer needed activations of the secondary pressure source, which reduces the number of these instances of volatility.

It is expected that similar pressure management improvements would be made when treating multiple wounds with a dual lumen system connected to multiple wounds using connectors (e.g., y-connectors) with continuous leaks adjacent the dressing of each of the multiple wounds being treated.

The foregoing description of preferred embodiments for this disclosure have been presented for purposes of illustration and description. They are not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated. All such modifications and variations are within the scope of the disclosure as determined by any future claims when interpreted in accordance with the breadth to which they are fairly, legally, and equitably entitled.

What is claimed is:

1. A system for managing reduced pressure to a wound site, the system comprising:
a wound enclosure configured to form a substantially sealed volume around the wound site;
a reduced pressure source in fluid communication with the wound enclosure, the reduced pressure source operable to apply reduced pressure to the wound enclosure for delivering exudate collected from the wound site to a canister through a first lumen;
a secondary pressure source in fluid communication with the wound enclosure operable to apply a secondary pressure to the wound enclosure through a second lumen to facilitate the flow of the exudate from the wound site to the canister through the first lumen; and
a vent disposed adjacent the wound site that is open to the atmosphere for introducing ambient air into the wound enclosure through the vent.

2. The system of claim 1 wherein the wound enclosure includes an enclosure interface having a dome and a flange disposed around the dome, the first lumen and the second lumen fluidly connected to the dome and the vent being disposed in the flange.

3. The system of claim 1 wherein the vent is operable to introduce a continuous flow of ambient air into the would enclosure.

4. The system of claim 3 further comprising a controller configured to communicate with the secondary pressure source for selectively activating the secondary pressure source to apply the secondary pressure to the wound enclosure while the vent introduces the continuous flow of ambient air into the wound enclosure.

5. The system of claim 1 further comprising a pump unit, the pump unit including the reduced pressure source and the secondary pressure source.

6. The system of claim 5 wherein the secondary pressure source includes a valve that is configured to be selectively opened to the atmosphere for applying the secondary pressure to the wound enclosure.

7. The system of claim 5 wherein the pump unit further includes a first pressure sensor for measuring a pressure of a fluid flow along a first fluid flow path that includes the first lumen and a second pressure sensor for measuring a pressure of a fluid flow along a second fluid flow path that includes the second lumen.

8. The system of claim 7 further comprising a controller operable to receive and compare pressure readings from the first pressure sensor and the second pressure sensor and selectively activate the secondary pressure source based on the comparison of the pressure readings.

9. The system of claim 7 further comprising a controller operable to receive and compare pressure readings from the first pressure sensor and the second pressure sensor and determine an operating condition of the system selected from a group consisting of a blockage condition, a canister fill condition, and a bolus condition.

10. A system for managing reduced pressure to a wound site, the system comprising:
a wound enclosure configured to form a substantially sealed volume around the wound site;
a pump unit including:
a reduced pressure source in fluid communication with the wound enclosure along a first fluid flow path, the reduced pressure source operable to apply reduced pressure to the wound enclosure for delivering exudate collected from the wound site to a canister through a first lumen,
a secondary pressure source in fluid communication with the wound enclosure along a second fluid flow path, the secondary pressure source operable to apply a secondary pressure to the wound enclosure through a second lumen to facilitate the flow of the exudate from the wound site to the canister through the first lumen,
a first pressure sensor in fluid communication with the first fluid flow path for measuring a pressure along the first fluid flow path,
a second pressure sensor in fluid communication with the second fluid flow path for measuring a pressure along the second fluid flow path, and
a controller operable to receive and compare pressure readings from the first pressure sensor and the second pressure sensor and selectively activate the secondary pressure source based at least in part on the comparison of the pressure readings; and
a vent disposed adjacent the wound site that is open to the atmosphere for introducing a continuous flow of ambient air into the would enclosure.

11. The system of claim 10 wherein the wound enclosure includes an enclosure interface having a dome and a flange disposed around the dome, the first lumen and the second lumen fluidly connected to the dome and the vent being disposed in the flange.

12. The system of claim 10 wherein the controller is configured to communicate with the secondary pressure source for selectively activating the secondary pressure source to apply the secondary pressure to the wound enclosure while the vent introduces the continuous flow of ambient air into the wound enclosure.

13. The system of claim 10 where the secondary pressure source includes a valve that is configured to be selectively opened to the atmosphere by the controller for applying the secondary pressure to the wound enclosure.

14. The system of claim 10 wherein the controller is operable to determine an operating condition of the system based at least in part on the comparison of the pressure readings from the fist pressure sensor and the second pressure sensor, the operating condition selected from a group consisting of a blockage condition, a canister fill condition, and a bolus condition.

15. A method of delivering reduced pressure to a wound site, the method comprising:
applying reduced pressure to a wound enclosure for delivering exudate collected from the wound site to a canister through a first lumen;
providing a secondary pressure source in fluid communication with the wound enclosure that is operable to apply a secondary pressure to the wound enclosure through a second lumen to facilitate the flow of the exudate from the wound site to the canister through the first lumen when the secondary pressure source is activated; and
continuously introducing ambient air into the wound enclosure through a vent disposed adjacent the wound site.

16. The method of claim 15 wherein the secondary pressure source is operable to be selectively activated to apply the secondary pressure to the wound enclosure while the vent is continuously introducing ambient air into the wound enclosure.

17. The method of claim 15 further comprising providing a pump unit, wherein the pump unit includes the reduced pressure source and the secondary pressure source.

18. The method of claim 17 wherein the secondary pressure source includes a valve that is configured to be selectively opened to the atmosphere for applying the secondary pressure to the wound enclosure.

19. The method of claim 15 further comprising:
determining a pressure of a first fluid flow path that includes the first lumen;
determining a pressure of a second fluid flow path that includes the second lumen;
determining a pressure differential between the pressure of the first fluid flow path and the pressure of the second fluid flow path; and
selectively activating the secondary pressure source based on the determination of the pressure differential between the first fluid flow path and the second fluid flow path.

20. The method of claim 19 further comprising determining an operating condition of the system selected from a group consisting of a blockage condition, a canister fill condition, and a bolus condition based at least in part on the pressure differential between the pressure of the first fluid flow path and the pressure of the second fluid flow path.
